(19) United States
(12) Patent Application Publication
(10) Pub. No.: US 2013/0013065 A1
(43) Pub. Date: Jan. 10, 2013

(54) TENDON REPAIR DEVICE AND METHOD

(76) Inventor: Matthew Bills, Provo, UT (US)

(21) Appl. No.: 13/541,586

(22) Filed: Jul. 3, 2012

Related U.S. Application Data
(60) Provisional application No. 61/505,316, filed on Jul. 7, 2011.

(51) Int. Cl. A61F 2/08

(52) U.S. Cl. 623/13.15

(57) ABSTRACT

A surgical device for assisting in the repair and rehabilitation of a tendon, formed of a cylindrical, helically wound braid (similar to a “Chinese finger trap”). The device may be implantable, and configured to enclose the repair site of the severed tendon. As tension or pull is applied to the tendon, the device tightens to secure the repair site. The device may improve mobility and use of the tendon by providing stability to the repair site, improving glide through the tendon sheath, allowing diffusion of nutrients to the repair site, and/or reducing adhesions within the tendon sheath.

### PRIORITY CLAIM

The present application claims priority to U.S. Provisional Patent Application Ser. No. 61/505,316, filed Jul. 7, 2011, which is expressly incorporated herein in its entirety.

### BACKGROUND OF THE INVENTION

1. The Field of the Invention

The present invention relates to a surgical device and method for treating damaged or severed tendons. More specifically, the present invention relates to an implantable device into which the tendon is inserted to facilitate surgical repair, stabilize the repair site, improve rehabilitation of damaged tendons, etc.

2. State of the Art

Tendon injuries are common and occur in a variety of situations including workplace accidents, auto accidents, and kitchen injuries. Tendon injuries cause many associated problems such as pain, reduced mobility, and reduced use of the affected body parts. Thus, it is desirable to treat tendon injuries promptly.

Tendon injuries may require multiple surgeries to correct. Additionally, the surgical process to repair tendons is often tedious and difficult. Flexor tendons may be located within a fibrous tendon sheath. The tendon sheath is highly specialized tissue that is anchored to the bone and is substantially hollow, forming a very smooth, tight “tunnel” around the tendon. It is the tendon sheath that ensures the tendon is in the proper place to effectuate movement at the appropriate joint. The sheath is a continuous tunnel, but also includes a plurality of discrete fibrous segments referred to as pulleys. The pulleys of the tendon sheath ensure the flexor pull of the tendon translates into joint motion. By approximating the tendons close to the bone, the pulleys of the tendon sheath provide a strong mechanical advantage when the joint is flexed, and also prevent bow-stringing of the tendon away from the bones.

When a tendon is lacerated, the proximal end of the tendon often retracts away from the injury site due to the action of muscles on the tendon. The surgeon must find both ends of the tendon, pull the ends through the tight tendon sheath and pulleys, and connect the two ends. Because tendons may be frayed and swollen, it is extremely difficult to manipulate the tendon through the sheath. Even if the surgeon is able to pull the tendon to the appropriate location and repair the laceration, the repair site is often bulky and/or rough. Thus, the repair site will abut the edges of the pulleys, making glide of the repair site through the tendon sheath difficult. Often, with each pass of the tendon through the pulley, abutment of the repair site against the pulley causes damage or irritation to the repair site, delaying the healing process.

Reduced glide of the tendon through the tendon sheath also leads to fibrosis and adhesions around the repair site. Often a second surgery is needed to release the fibrosis and adhesions, in addition to the first tendon repair surgery.

It is believed that early mobility and controlled mechanical strain facilitate healing of the tendon. While stretching can disrupt healing during the initial inflammatory phase, studies have shown that controlled movement of the tendons within the first week following an acute injury can help promote synthesis of collagen by tendon cells, leading to increased tensile strength and diameter of the healed tendons and fewer adhesions compared to tendons that are immobilized. To further show that movement and activity assist in tendon healing, studies have been conducted where the tendons are immobilized after injury, and there has been a negative effect on healing. It is thus desirable to repair a tendon with sufficient strength to allow early mobility, while at the same time, limiting handling of the injured tendon and decreasing operation time.

While movement and some mechanical strain facilitate healing, over-aggressive movement of a tendon after surgery tends to pull the repair site apart, potentially implicating another tendon repair surgery. It can be difficult for patients with tendons healing from repair surgery to effectuate the proper movement and activity to improve healing, while not causing any aggressive movements that might damage the healing tendons. Thus, there is a need for an improved device and method for tendon repair surgery which may allow for early mobilization and mechanical strain while providing support and reducing risk of reinjury to thereby expedite healing of the tendon.

### SUMMARY OF THE INVENTION

It is an object of the present invention to provide a device for facilitating the repair and healing of damaged or severed tendons. It is another object of the present invention to provide a method for facilitating repair and healing of damaged or severed tendons.

In accordance with the principles of the present invention, a tendon repair device and method are disclosed which facilitate movement and healing in a tendon. Because damaged tendons may become frayed and swollen in vivo, and thus difficult to manipulate to a desirable position within the tight tendon sheath, the present invention may facilitate manipulation of the severed tendon through the sheath during surgery.

In accordance with another aspect of the invention, the tendon repair device and method may facilitate early mobilization of the repaired tendon.

According to one aspect of the invention, the device constricts the repair site such that tendon glide within the tight tendon sheath is facilitated.

According to another aspect of the invention, healing may be further facilitated by constructing the device such that nutrients may diffuse in and around the healing tendon while still providing support to the injured portion of the tendon.

According to another aspect of the invention, the device is constructed to at least partially contain the frayed ends of the tendon, thereby decreasing the chance for scar tissue adhesion within the tendon sheath.

According to the present method, the device may be deployed during surgery by inserting each end of a severed tendon into the device such that the two ends meet within the device. When tension is applied to the tendon, the device tightens, thus holding the severed ends of the tendon even more securely and facilitating early mobilization, glide, and reducing adhesions.

These and other aspects of the present invention are realized in the device and method as shown and described in the following figures and related description.

### BRIEF DESCRIPTION OF THE DRAWINGS

Various embodiments of the present invention are shown and described in reference to the numbered drawings wherein:

FIG. 1A shows a perspective view of a tendon repair technique according to the prior art;

FIG. 1B shows a perspective view of a tendon repair technique according to the prior art;

FIG. 2 shows a perspective view of a bulky repair site abutting the edge of a pulley of the tendon sheath as the tendon glides during joint movement, according to the prior art;

FIG. 3 shows a perspective view of the device according to the present invention;

FIG. 4A shows a magnified view of the warp and weft threads of the device and an example of their associated angles when the device is not under tension;

FIG. 4B shows a magnified view of the warp and weft threads of the device and an example of their associated angles when the device is under tension;

FIG. 5 shows a representation of a cross-section view of the device and its circumferences relaxes and under tension;

FIG. 6 shows a magnified view of the weaving employed in the device;

FIG. 7 shows a partially transparent view of the device in vivo, with a repaired tendon laceration located within the device;

FIG. 8A shows a perspective view of the device according to the present invention, as in use on a lacerated tendon;

FIG. 8B shows a diagram of the radius of the device of FIG. 8A;

FIG. 8C shows a perspective view of the device according to FIG. 8A when the device experiences pull or tension;

FIG. 8D shows a diagram of the radius of the device in FIG. 8C under pull or tension;

FIG. 9A shows a side view of a one aspect of the invention wherein the device has one flared end; and

FIG. 9B shows a side view of a one aspect of the invention wherein the device has two flared ends.

It will be appreciated that the drawings are illustrative of various aspects and embodiments of the present invention and not limiting of the scope of the invention which is defined by the appended claims. The embodiments shown accomplish various aspects and objects of the invention, though it is not necessary that any embodiment accomplish all aspects or any particular aspect of the invention. It is appreciated that it is not possible to clearly show each element and aspect of the invention in a single figure, and as such, multiple figures are presented to separately illustrate the various details of the invention in greater clarity. It will be understood that various structures shown in one figure may be used in a device shown in another figure.

### DETAILED DESCRIPTION

The invention and accompanying drawings will now be discussed in reference to the numerals provided therein so as to enable one skilled in the art to practice the present invention. The drawings and descriptions are exemplary of various aspects of the invention and are not intended to narrow the scope of the accompanying claims.

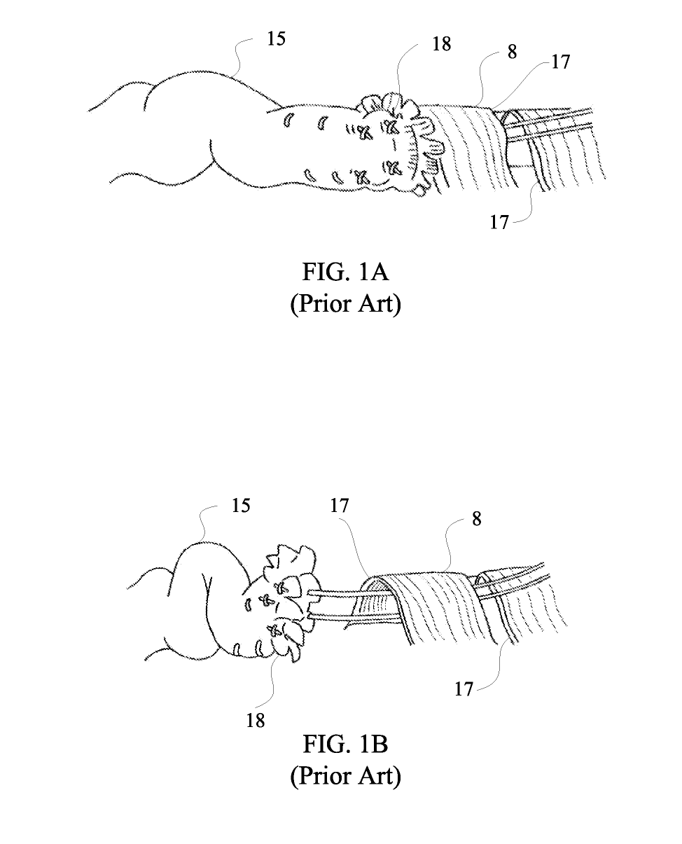

FIGS. 1A and 1B show a severed tendon and a method of repairing it according to the prior art. The prior art demonstrates many of the problems related to tendon repair. For example, a tendon 15 may have an inflamed or frayed end 18. Because the inflamed or frayed end 18 has increased in size relative to an uninjured tendon, the frayed end 18 is difficult to thread through the tight pulley 8 of the tendon sheath. As shown in FIG. 1B, forcing the inflamed or frayed end 18 through the pulley 8 may cause further damage to the tendon end 18.

The two ends of the tendon (only one of which is shown in FIGS. 1A and 1B) may be sewn back together. However, the rejoined tendon has an inflamed or frayed portion which is thicker than the rest of the tendon and is more likely to catch on the edge 17 of the pulley 8 or irritate the pulleys 8 of the tendon sheath.

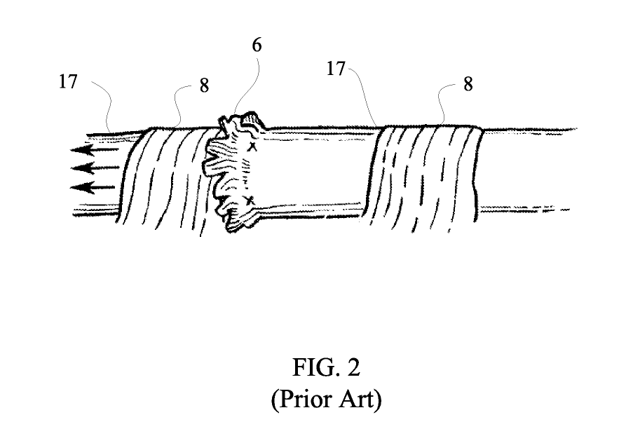

As shown in FIG. 2 the repaired tendon may be significantly inferior to the tendon prior to the injury. Even if the surgeon is able to successfully repair the tendon, the repair site 6 is often bulky. Further damage can be caused to the repair site as it abuts the edge 17 of the pulley 8. For example, frayed ends may catch on the pulleys, causing more resistance to movement and increasing the risk of further damage to the tendon. This damage and pulling of the repair site often precipitates the need for additional repair surgeries. While movement and physical therapy is desirable in facilitating healing of the wound, sometimes these very movements can cause disrepair and harm to the site. The nature of the tendon sheath and the necessity of the repair site to move freely within the sheath present a unique problem in the healing process of injured tendons.

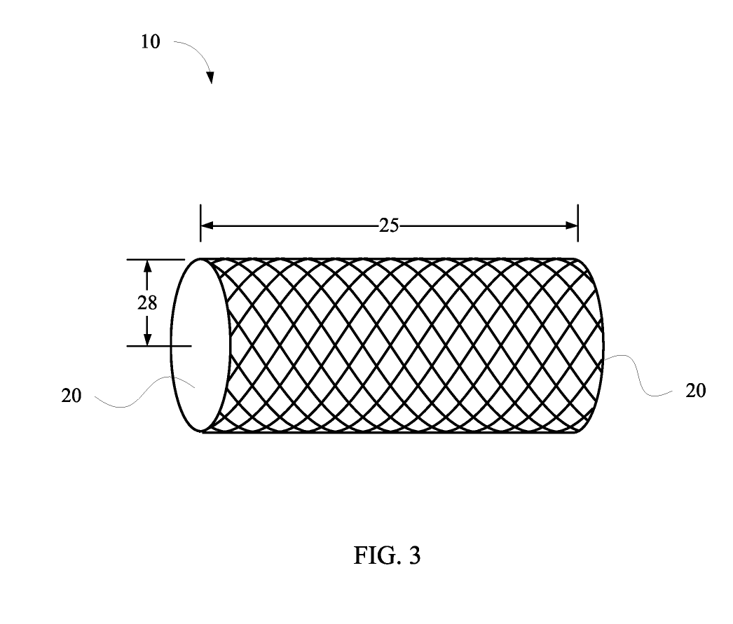

Turning now to FIG. 3, there is shown a perspective view of a tendon repair device, generally indicated at 10, which may help to brace the tendon and thereby facilitate both surgical repair and subsequent healing of the tendon. The tendon repair device 10 includes a body which approximates or functions in a manner somewhat similar to a “Chinese finger trap” in structure and concept and may provide additional support to the tendon when placed under mechanical strain.

The body of the tendon repair device 10 may be generally cylindrical (or other cross-sectional shape similar to that of a tendon) and has two opposing open ends 20, a length 25, and a radius 28. The tendon repair device 10 may be formed of a helically wound braid, such as a biaxial braid, for example. The braid may be formed from a number of pieces of material which are wound together, or from a single wound piece of material. The material may be selected from a variety of biocompatible materials, including material commonly used for sutures. Depending on the needs, the repair device may be formed from a dissolvable material, such that it will be absorbed by the body after a period of time, or a more permanent material.

In use, the tendon repair device may provide stability to the repair site as it is moved and stretched, because the repair device constricts as it is lengthened. As the tendon repair device 10 is pulled in opposing directions, the length 25 increases and the radius 28 decreases. This effects a reduction in the volume along any given length of the tendon repair device and tightens its engagement with a tendon disposed therein based on the normal behavior of a cylindrical, helically wound braid. Any pulling on the tendon, and thus device, lengthens and narrows the device, thereby providing increased support around the injured portion of the tendon.

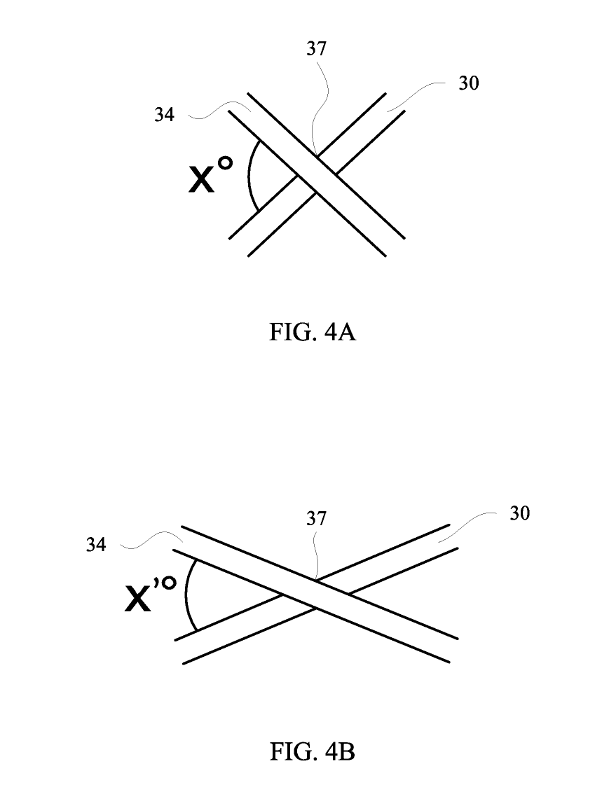

FIGS. 4A and 4B show magnified views of the weaving of the material which makes up the body of the tendon repair device. When the device is stretched, the length of the device is gained by reducing the angle x between the warp 30 and weft 34 threads at their crossing points 37. In FIG. 4A, no tension is applied to the device and the angle x may be, for example, approximately 90 degrees. In FIG. 4B, the device is subject to a pulling, and the angle x′ has decreased to, for example, approximately 45 degrees. The more pulling or tension applied to the device, the more the circumference and diameter shrink (i.e., the device tightens). These particular angles are exemplary, and one of skill in the art would appreciate that the tendon repair device may be formed to create a variety of angles x and x′ between the warp 30 and weft 34 threads at their crossing points 37, depending on the desired results for a particular situation.

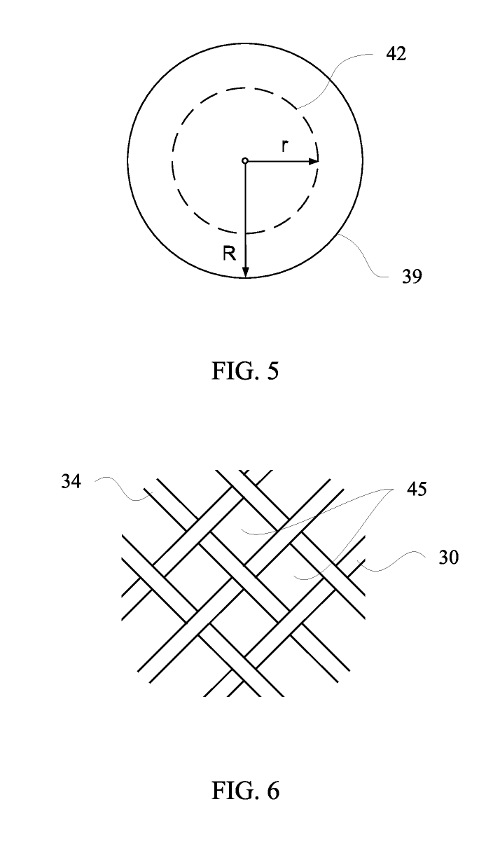

At the same time the length 25 of the device is increased, the radial distance between opposing sides and hence the overall circumference and diameter are reduced. FIG. 5 shows a representation of a cross-sectional view of the device. The solid circle 39 indicates the circumference when no tension is applied to the device. Without tension, the device as a radius R as indicated. The dashed circle 42 indicates the contracted circumference of the device when it is under tension. Under such tension, the radial distance is decreased and the device has a smaller radius r as indicated. It will be appreciated that the change in the radius shown in FIG. 5 is for demonstration purposes only and is not meant to show the proportional reduction in the radius. Those skilled in the art will appreciate that the tendon repair device will preferably reduce in diameter sufficiently to add additional support and constrict any inflamed portion of the tendon contained therein, but not constrict so significantly so as to place excessive constrictive pressure on the tendon. Thus, the diameter of the device may reduce from between about 5 and 50 percent, with a reduction in the diameter of between about 5 and 15 percent being presently preferred.

The device may be constructed of different circumferences to accommodate different sized tendons. Preferably, the device would come in a few standard sizes that would cover the range of nearly any tendon. Additionally, specially-sized devices could be made.

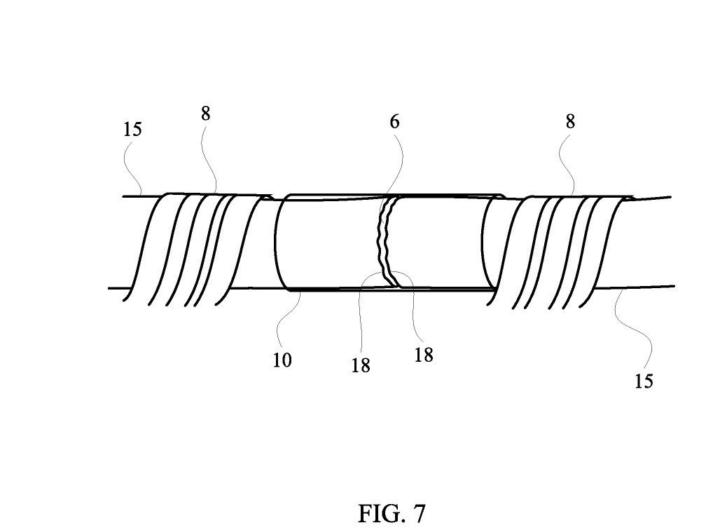

Turning now to FIG. 6, a magnified view of the weaving according to the present device is shown. Gaps 45 are left between the warp 30 and weft 34 threads as woven in the device to facilitate the flow of fluid into contact with the tendon. This design of the tendon repair device will allow for diffusion of nutrients in and around the repair site 6, while at the same time minimizing adhesions and fibrosis around the damaged area. While FIG. 6 depicts the weaving of the device as a simple weave, one of skill in the art will appreciate that other types of weaves could be used, such as a twill weave, for example.

The device may be constructed of any suitable biocompatible, surgically implantable material known in the art. For example, the device may be made of a material that is biodegradable, such as those that are currently known in the medical field. This would allow the material to gradually disappear as the tendon heals. The device may also be made of a polymer commonly used as suture material, for example, 4-0 polypropylene, or 4-0 nylon suture material (such as ETHILON®) may be used. Other sizes and materials may be used as well. Additionally, the body of the device may be formed from multiple pieces of polypropylene and/or nylon, which are fused together, or from a single wound piece of suture material.

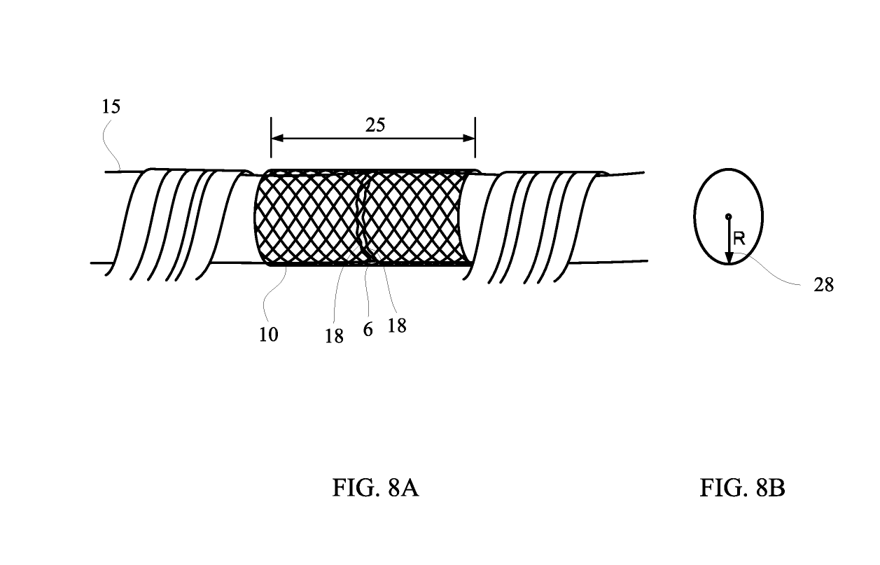

Turning now to FIG. 7, a partially transparent view of the device in use on a tendon repair site is shown so as to reveal the juncture of the torn tendon. In use, the surgeon would insert each end 18 of a severed tendon 15 into the tendon repair device 10 so that the two ends meet within the device. (If necessary, the surgeon may trim frayed ends of the tendon.) The surgeon may slide the tendon repair device 10 over one end of the injured tendon 15, suture the ends 18 of the tendon together, and then slide the tendon repair device over the sutured ends and add one or more sutures to help hold the tendon repair device in place over the sutured ends. In the alternative, the ends 18 may be simply advanced into the tendon repair device 10 until they meet and then the surgeon may add one or more sutures used to hold the tendon repair device to each side of the severed tendon 15. Specific instrumentation may be used to deploy the tendon repair device. For example, instrumentation to hold the ends of the tendon, to advance and position the device, to hold the repaired segment until sutures are placed, etc. The tendon repair device 10 can be used in conjunction with other instrumentation known in the art in facilitating tendon repair. The tendon repair device 10 may be sutured in place by, for example, 6-0 polypropylene or nylon suture or another suitable material known in the art.

In either method, the tendon repair device 10 may help to hold the tendon ends 18 together while they heal, and allow portions of the tendon proximal and distal from the injury to bear some of the load when the tendon is placed under mechanical strain. Thus, a patient may be able to move the tendon and place some strain thereon, thereby obtaining the benefits of mechanical strain on the injured tendon with less risk of re-injury.

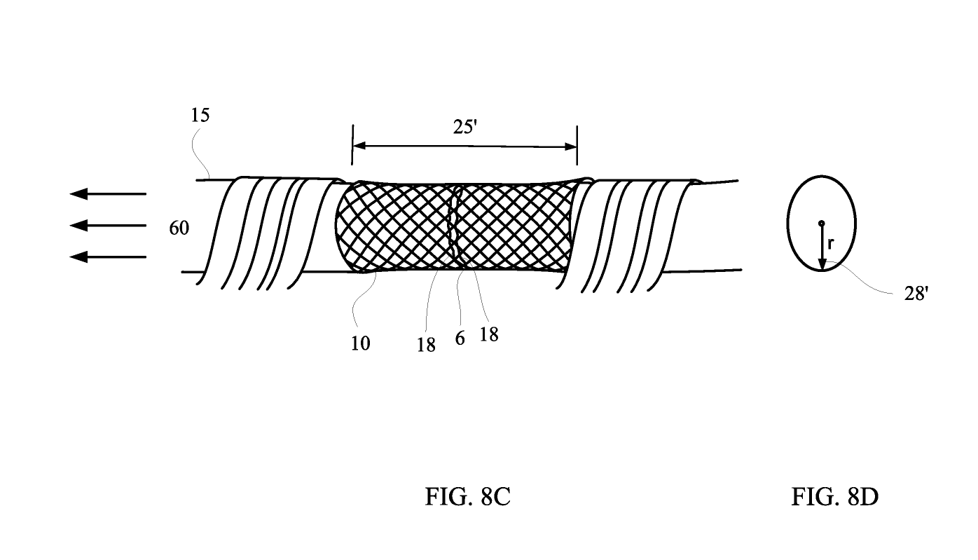

FIG. 8A shows the tendon repair device 10 as employed by either of the method described above on a tendon repair site 6. In FIG. 8A, there is no tension on the tendon 15 and the tendon repair device 10 has its standard at-rest length 25 and radius 28. FIG. 8B shows a representation of a cross-sectional view of the tendon repair device 10 as shown in FIG. 8A, to demonstrate the at-rest radius, R, of the device.

Turning now to FIG. 8C, there is shown a side view of the device and repair site shown in FIG. 8A, but now with the tendon 15 being placed under tension as indicated by the arrows 60. As tension 60 is applied to the tendon 15 which would tend to pull the repair site 6 apart, the tension 60 causes the device 10 to tighten, with length 25′ increasing as the radius 28′ decreases, thus holding the severed ends 18 even more securely and limiting their ability to pull away from each other. FIG. 8D shows a representation of a cross-sectional view of the device in FIG. 8C under tension, and its accompanying radius 28′ with a length r. One having skill in the art will appreciate that the length 25 and the increased length 25′ are both shown by way of example, and that numerous different lengths and increased lengths would be possible and within the scope of the present invention. Similarly, the radius 28 and the reduced radius 28′ are shown as examples for illustrative purposes, and varying radii and reduced radii would be possible according to the present invention, given the size of the device used and the amount of strain placed on the device. While it may be helpful to provide the device in several standard lengths and radii, the device may also be specially constructed according to the specific needs of a particular patient.

As the patient works to gain mobility and perform physical therapy for the injured tendon to facilitate healing, the tendon repair device 10 will create further stability for the tendon. Any movement of the patient that tended to pull the repair site 6 apart would cause the device 10 to tighten and stabilize the repair site 6.

Additionally, the device 10 is low-profile, and one of skill in the art would appreciate that as the device tightens, it occupies an even smaller cross sectional area. Because the device 10 contains any frayed ends or bulky repair site, there is a reduction in the frayed ends and/or bulky repair site abutting the edge of a pulley within the tendon sheath. This may increase tendon glide for early mobilization. Furthermore, fibrosis and scar tissue adhesion to the tendon sheath are reduced because the frayed ends of the tendon 18 are located within the device 10. The device is also quickly deployed in surgery, allowing for a shorter operative time, limited exposure, and decreased manipulation of the tendon and sheath.

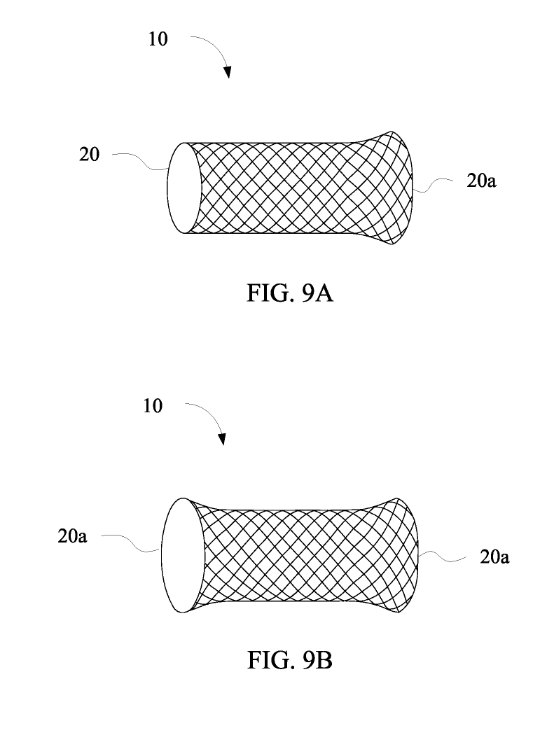

Additional embodiments of the current device could be used. For example, the device could have a flared end on either one end or both ends. Thus, as shown in FIG. 9A, the tendon repair device 10 may have a flared, distal end 20a. Another possible embodiment is shown in FIG. 9B, with both the proximal end and the distal end flared, 20a. The flared ends may help to direct the ends 18 of the tendon 15 (FIG. 8A) into the tendon repair device 10. The flared ends may be formed by weaving the device more loosely at one end or both ends 20a. The flared ends could be tightened or constricted by anchor sutures that extended from the weave and could be pulled to constrict the ends. The anchor sutures could then be sewn down and serve as anchor points at a position distal from the main juncture of the torn tendon.

Different configurations may be desired where the surgeon requires additional assistance in threading the severed ends of the tendon through the pulley, or when the severed ends of the tendon are to be joined within a pulley. Likewise, the device could be formed to wrap around a tendon which has been damaged but not completely severed. For example, the device may be split along its length such that a tendon or ligament could be inserted within the device. The device may also be used in settings other than damaged tendons, such as for damaged ligaments, or for attaching a tendon to a bone, etc.

There is thus disclosed an improved device for treating and healing injured tendons. It will be appreciated that numerous changes may be made to the present invention without departing from the scope of the claims.

What is claimed is:

1. A device for attaching a two portions of a tendon to one another, the device comprising: an elongated hollow member of woven construction of a biocompatible, surgically implantable material and substantially tubular shape, the member having a first end and a second end, the device being open on the first end and the second end as a first diameter when in a relaxed state, the woven construction being oriented such that pulling longitudinally on opposing ends of the elongated hollow member causes the woven construction to realign and reduce the diameter of the elongate hollow member into a second diameter smaller than the first diameter.

2. A device for assisting in the repair or rehabilitation of a damaged or severed tendon, the device comprising: a body formed from interwoven fibers of a biocompatible, surgically implantable material, the body circumscribing a hollow portion and having an open first, proximal end and an open second, distal end.

3. The device of claim 2, wherein the body formed from interwoven fibers is sized to receive and hold a tendon.

4. The device of claim 2, wherein the body formed from interwoven fibers is sized to receive and hold a ligament.

5. The device according to claim 2, wherein the body forms a flared opening at at least one of the first, proximal end and the second, distal end.

6. The device according to claim 5, wherein the flared opening is formed by a looser weaving of the interwoven fibers at at least one of the first, proximal end and the second, distal end.

7. The device of claim 2, wherein the body is formed from suture material.

8. The device of claim 7, wherein the body is formed from a single piece of suture material.

9. The device of claim 7, wherein the body comprises multiple pieces of suture material fused together at the ends.

10. The device of claim 2, wherein the body formed from interwoven fibers comprises a braid, the body having a radius, and the braid being wound such that pulling on the braid reduces the radius of the body.

11. The device of claim 10, wherein the braid is a cylindrical, helically wound braid.

12. The device of claim 2, wherein the body formed from interwoven fibers further comprises a length, the length extending between the first, proximal end, and the second, distal end, the device being split along the length of the body.

13. A method for treating damaged or severed tendons or ligaments, the method comprising:
selecting a body formed by a woven generally hollow member having an open first end and an open second end;
selecting severed ends of a tendon or ligament; and
advancing the severed tendon or ligament ends into the open end first end and the open second end of the body and securing the body to the tendon or ligament so that the severed ends of the tendon or ligament remain in the body.

14. The method of claim 13, further comprising the step of suturing the body to the tendon or ligament on at least one side of the severed ends.

15. The method of claim 14, wherein the method comprises suturing the body to the tendon or ligament on both sides of the severed ends.

16. The method according to claim 13, wherein selecting a body comprises selecting a woven body with a radius, the body being woven such that pulling on the body causes the body of the radius to reduce.

17. A method of assisting in the repair and healing of damaged or severed tendons or ligaments, the method comprising:
selecting a body formed by a woven generally hollow member having an open first end and an open second end, the body having a length extending from the open first end to the open second end, the body being split along the length;
selecting a damaged or severed tendon or ligament; and
advancing the damaged or severed tendon or ligament into the split along the length of the body and securing the body to the tendon or ligament so that the damaged or severed tendon or ligament remains in the body.

18. The method of claim 17, further comprising the step of suturing the body to the tendon or ligament on at least one side of the severed or damaged tendon or ligament.

19. The method of claim 17, wherein the method comprises suturing the body to the tendon or ligament on both sides of the severed or damaged tendon or ligament.

20. The method according to claim 17, wherein selecting a body comprises selecting a woven body with a radius, the body being woven such that a pulling force along the length of the body causes the body of the radius to reduce.

21. The method according to claim 17, wherein the method further comprises the step of closing the split along the length of the body after the damaged or severed tendon or ligament has been advanced into the body and secured to the body.

22. The method according to claim 17, wherein the selecting a body comprises selecting a woven body comprised of a braid, the body having a radius, and the braid being wound such that pulling on the braid reduces the radius of the body.

23. The method according to claim 22, wherein the selecting a body further comprises selecting a woven body with a weaving that is looser on at least one of the open first end and the open second end, the looser weaving thus forming a flared opening at at least one of the open first end and the open second end.

24. The method according to claim 23, wherein the method further comprises the step of suturing at least one of the open first end and the open second end to a point away
